(19) United States
(12) Patent Application Publication
(10) Pub. No.: US 2020/0046782 A1
(43) Pub. Date: Feb. 13, 2020

(54) Transient Commensal Microorganism for Improving Gut Health

(71) Applicant: EVOLVE BIOSYSTEMS INC., Davis, CA (US)

(72) Inventors: David KYLE, Davis, CA (US); Samara FREEMAN-SHARKEY, Davis, CA (US); Steven FRESE, Oakland, CA (US)

(21) Appl. No.: 16/084,154

(22) PCT Filed: Mar. 13, 2017
(86) PCT No.: PCT/US17/22209

§ 371 Date: Sep. 11, 2018

Related U.S. Application Data
(60) Provisional application No. 62/307,420, filed on Mar. 11, 2016.

(51) Int. Cl. A61K 35/745; A61K 31/702; A61K 9/14

(52) U.S. Cl. CPC A61K 35/745; A61K 35/747; A61K 9/14; A61K 31/702

(57) ABSTRACT

The inventions described herein relate generally to administering compounds to promote mucosal healing in mammals in need thereof including, but not limited to humans. The compounds may include bifidobacteria and mammalian milk oligosaccharides.

### FIELD OF THE INVENTION

The embodiments described herein relate generally to healthcare, and more particularly, to administering compounds to promote mucosal healing in mammals in need thereof including, but not limited to humans.

### BACKGROUND

The intestinal microbiome is the community of microorganisms that live within the gastrointestinal tract, the majority of which is found in the large intestine or colon. In a healthy individual, most dietary nutrients that are consumed are absorbed by the body before they reach the colon. Many foods, however, contain indigestible carbohydrates (i.e dietary fiber) that remain intact and are not absorbed during transit through the gut to the colon. The colonic microbiome comprises certain bacterial species that are able to partially consume these fibers and utilize the constituent sugars (free sugar monomers or FSMs released by microbial digestion of the fibers) for energy and metabolism, as well as a larger number of bacterial species that simply thrive on the FSMs produced by these fiber degraders. Methods for measuring dietary fiber in various foods are well known to one of ordinary skill in the art.

In mammalian species, the nursing infant's intestinal microbiome is quite different from that of an adult microbiome in that the adult gut microbiome generally contains a great diversity of organisms all present in a low percentage of the total population. The nursing human infant's microbiome, on the other hand, can be made up almost exclusively (up to 80%) of a single species. Diet drives the abundance, complexity and diversity of microbial species in the microbiome. The abundance of different species in the colonic microbiome is the result of the diversity of the fiber in the diet of a typical adult whereas the nursing infant has only a single source of dietary fiber—mammalian milk oligosaccharides (MMOs)—and hence the resultant infant microbiome can be dominated by organism(s) that preferentially utilize that type of dietary fiber.

The transition from the simple, non-diverse microbiome of the nursing infant to a complex, diverse microbiome of an adult reflects the mammal's transition from a single nutrient source of a complex fiber (e.g, mammalian milk oligosaccharides—MMOs) to more diverse dietary fiber sources. The post-weaning through adult mammalian microbiome contains a more diverse number of microbial species able to compete in the variable food niches that are generated by the diversity of the fibers in the complex diet of an adult relative to that of the infant.

Pre-weaned mammalian infants have only one source of nutrition: mammalian milk. Components in mammalian milk, namely mammalian milk oligosaccharides, have, over the course of evolution, selected for a small number of organisms that are particularly suited to the intestinal environment, grow selectively on MMO, and confer benefits to the host. In the case of the breastfed infant, the indigestible portion of the milk is effectively broken down and consumed by these selected organisms. As a result, these organisms are able to outcompete and increase their abundance compared to other environmental species, and this has the overall effect of reducing complexity of the microbiome.

For example, the HMOs represent about 15% of total dry weight (and energy) and are the third most abundant family of nutrients in human milk. These oligosaccharides comprise sugar residues in a complex and branched form that is not usable directly as an energy source for the baby or an adult, or for most of the microorganisms in the gut of that baby or adult. A distinct few microorganisms, such as Bifidobacterium longum subsp. infantis (B. infantis), and Bifodobacterium breve, have the unique capability to consume specific MMOs, such as those found in human or bovine milk (see, e.g., U.S. Pat. No. 8,198,872 and U.S. patent application Ser. Nos. 13/809,556 and 62/307,425, the disclosures of which are incorporated herein by reference in their entireties). When B. infantis comes in contact with certain mammalian milk oligosaccharides, a number of genes are specifically induced which are responsible for the uptake and internal deconstruction of those mammalian milk oligosaccharides, and the individual sugar components are then catabolized to provide energy for the growth and reproduction of that organism (Sela et al, 2008).

If the appropriate bacteria are not present in the body of the mammal, the indigestible carbohydrates of, for example, mammalian milk become susceptible to non-specific hydrolysis, releasing FSMs capable of promoting the growth of opportunistic or highly destructive pathogens that would not have flourished otherwise, or are otherwise excreted from the body in the feces. The consequence of a dysbiotic microbiome is that is skewed towards infection, inflammation, intestinal damage, and pathogenesis.

Conventional teaching with regards to the mammalian microbiome is that complexity provides stability. To be able to effectively consume the complex non-infant diet, maintaining a diversity of microorganisms in the microbiome is thought to be the key to promoting gut health. Lozupone, Nature, Vol. 489, pp. 220-230 (2012). The inventors have discovered that this is not necessarily the case and that the simplest microbiome may be of great benefit to the stabilization and recovery of the gut damaged by inflammation and/or agitation.

### SUMMARY

The inventors have discovered that mucosal healing in the dysbiotic gut can be promoted by probiotic microorganisms driving the intestinal microbiome towards an infant-like state, which is simple, less diverse, and less pro-inflammatory than an adult gut microbiome particularly when inflamed by disease or dysbiosis. The inventors have also discovered that use of probiotic microorganisms for which MMOs serve as a selective energy source (carbon source), is particularly beneficial for mucosal healing. Dysbiotic mammals in need of mucosal healing would include humans that exhibit conditions such as, but not limited to, Irritable Bowel Syndrome (IBS), Crohn's Disease (CD), Ulcerative Colitis (UC) (collectively Irritable Bowel Disease or IBD), Short Gut Syndrome, colic, general diarrhea, overgrowth of certain pathogenic bacteria such as Clostridium difficile, or any other condition (such as extensive antibiotic use) where the gut is made susceptible to infection by pathogens, such as, but not limited to Escherichia, Clostridium, Shigella, Campylobacter, and Salmonella.

This invention provides methods of treating gastrointestinal dysbiosis by providing a patient with a composition comprising (i) a complex carbohydrate from a mammalian milk source and (ii) bifidobacteria which internalize the MMO prior to its hydrolysis; typically the composition is administered for a period of time (e.g., for at least 5 days). While the microorganisms and oligosaccharides are normal components of infant nutrition, the methods of this invention are targeted at subjects other than infants (i.e. beyond 6 months of life); therefore, the compositions of this invention are formulated for more mature (and typically larger) subjects and for compatibility with more complex diets. The dysbiosis may be the result of Irritable Bowel Syndrome, Crohn's Disease, Ulcerative colitis, Necrotizing Enterocolitis, bacterial overgrowth, bacterial induced diarrhea, antibiotic treatment, eating disorders, obesity, or low diversity in dietary intake. The Bifidobacterium is preferably selected from B. longum, B. pseudocatanulatum, B. adolescentis, B. animalis (e.g., B. animalis subsp. animalis, B. animalis subsp. lactis), B. longum subsp. longum, B. pseudolongum, and B. breve, and more preferably, the B. longum is B. longum subs. infantis. The mammalian milk sources may be human, caprine, porcine, ovine, equine, or bovine, and preferably, the bovine source is from bovine colostrum. The MMO may be from whey, whey mother liquor, whey powder, whey permeates, and/or the mammalian milk oligosaccharide may be fucosylated, sialylated or be derivatives thereof. In particular, the MMO may comprise 2′-fucosyllactose, 3′-fucosyllactose, difucosyllactose, lacto-N-fucosylpentose I, lacto-N-fucosylpentose II, lacto-N-fucosylpentose III, lacto-N-fucosylpentose V, 3′-sialyllactose, 6′-sialyllactose, 3′-sialyl-3-fucosyllactose, sialyllacto-N-tetraose, 3′-sialyllactoseamin, and 6′-sialyllactosamine, produced synthetically and purified or isolated from natural sources or recombinant microorganisms. The Bifidobacterium is typically provided in a daily dose of from 10 million to 1 trillion cfu, preferably 10 million to 100 billion cfu, and more preferably from 4 billion to 50 billion cfu. The MMO is provided in a daily dose of from 1 to 60 g, preferably from 2 to 40 g.

Some embodiments of the instant invention include compositions comprising a MMO and a microorganism wherein the MMO induces a change in the microorganism such that the MMO then becomes an energy source for the organism, and when ingested by a mammal, the induced or activated microorganism provides a benefit to the gut of that mammal. Additional embodiments involve the maintenance of the induced microorganism in the gut of the mammal by maintaining the dietary supply of MMOs or other glycans that are selective for that microorganism. A further embodiment involves the subsequent clearance of the microorganism from the gut by the cessation of the supply of the MMO to the mammal.

In one mode of this invention, the MMO and the bifidobacteria are provided in a dry form which may also be enrobed in a material that would provide enteric protection. In another mode, the MMO and the bifidobacteria are encapsulated, and the capsule may further comprise an enteric coating such as a coating that is not disrupted by passage through the stomach. In another mode, the MMO is provided as a solution and the bifidobacteria is provided as an enteric-coated powder, tablet, or capsule.

In yet another mode, the method of this invention further comprises administering Lactobacillus or Pediococcus contemporaneously with the composition. The Lactobacillus may be selected from L. plantarum, L. casei, L. antri, L. brevis, L. coleohominis, L. fermentum, L. gasseri, L. johnsonii, L. pentosus, L. sakei, L. salivarius, L. rhamnosus (e.g., LGG), L. acidophilus, L. curvatus, L. mucosae, L. crispatus, and/or L. reuteri. Preferably the Lactobacillus is L. reuteri. The Pediococcus may be selected from P. acidilactici, P. stilesii, P. argentinicus, P. claussenii and/or P. pentosaceus. The Lactobacillus or Pediococcus may be provided in a daily dose of from 10 million to 1 trillion cfu, preferably the Lactobacillus and Pediococcus is provided in a daily dose of from 5 billion to 50 billion cfu.

### BRIEF DESCRIPTION OF THE DRAWINGS

FIG. 1: Chart showing the base 10 log change in B. infantis levels by day during mucosal healing diet including BMO, GOS, and B. infantis. The data are reported as CFU B. infantis per ug DNA divided by CFU total bacteria per ug DNA.

FIG. 2: Chart showing the overall weight gain at 28 days for piglets receiving standard antibiotics at birth, no antibiotics or the mucosal healing preparation described in the application.

### DETAILED DESCRIPTION

### Development of the Neonatal Microbiome

Certain bifidobacteria, such as B. longum subsp. infantis, possess certain genes individually or in gene clusters that are dedicated to the internalization and deconstruction of HMOs (Sela and Mills, 2010, Trends in Microbiol., 18:298-307). When such bacteria interact with MMOs, like those found in mammalian milk, these genes for transporting and catabolizing fucosylated and/or sialylated oligosaccharides, are upregulated (Kim, et al., 2013, PLoS ONE, 8(2):e57535; Garrido, et al., 2015, Nature Scientific Reports). The inventors have recently discovered that certain bifidobacteria including, but not limited to B. infantis can be “activated” by their interaction with certain MMOs (International Patent Publication No. WO 2016/065324, incorporated by reference herein). The activated B. infantis is defined herein as the state of the cells, as measured by the up-regulation or down-regulation of certain genes including, but not limited to, oligosaccharide binding proteins, permeases, and enzymes responsible for the uptake and internal deconstruction of the MMO. In the activated form, the B. infantis becomes the primary consumer of all the MMO and has been shown to increase its relative proportion in the gut microbiota of breast-fed infant humans to levels significantly higher than its natural levels and as high as 70% of the total microbial population of the distal colon. When B. infantis is present in the gut of a baby, and that baby is also provided with mother's milk as a primary or sole source of nutrition, the population of B. infantis can increase to levels as high as 90% of the total bacterial population of the gut as measured by the microbial quantification of the stool. When present in this situation, many other genes are also upregulated including those for the production of a number of other metabolites.

When activated, B. infantis is known to bind tightly to the gut mucosa of the baby and facilitate the development of the infant gut (Underwood, et al., 2015, Pediatr. Res., 77:229-235). The proliferation of activated B. infantis in the gut of a newborn infant, triggered and uniquely enabled by the MMOs provided in mother's milk, is of significant benefit to the health and long term survival of that infant. B. infantis is associated with significant benefits to a newborn infant which include, but are not limited to, a higher binding affinity to the gut mucosa, higher colonization of the GI tract thereby preventing growth of other bacterial clades, higher consumption of MMOs, and a greater stimulation of the immune response (Lewis, et al., 2015, Microbiome, 3:13; Huda, et al., 2014, Pediatrics, 134:2 e362-e372).

Once administered with a sufficient amount of MMOs as a dietary source, the activated B. infantis will remain in the gut of a mammal at high concentrations and activated as long as the dietary source of MMOs is continuously provided to the mammal. The inventors have discovered that once the source of the MMOs is withdrawn from the diet (e.g., at weaning), the B. infantis is no longer activated, and it can no longer successfully colonize or compete with other gut microbiota for nutrients in the gut, and its population rapidly decreases to less than 5% of the total microbiome. B. infantis is generally not naturally found in the gut of a weaned infant, child, or adult in levels of more than 1%.

### Mammalian Milk Oligosaccharide Nutrients

For this invention, the MMOs are typically sourced from, identical to, or functional equivalents of those oligosaccharides in mammalian milks including, but not limited to, human, caprine, bovine, equine, or ovine milk. The term “mammalian milk oligosaccharide” (MMO), as used herein, refers to those indigestible glycans, sometimes referred to as “dietary fiber”, or the carbohydrate polymers which are not hydrolyzed by the endogenous host enzymes in the digestive tract and remain generally unabsorbed in the intestinal lumen (e.g., the small intestine) of the mammal. Although “dietary fiber” usually refers to indigestible plant polysaccharides with degree of polymerization (D.P.) of 20 or more carbohydrate residues, MMO includes branched-chain oligosaccharides and oligosaccharides between DP-3 and DP-20. Oligosaccharides may be free in milk or bound to protein or lipids and are also referred to as glycans. Oligosaccharides having the chemical structure of the indigestible oligosaccharides found in any mammalian milk are called “MMO” or “milk fiber” herein, whether or not they are actually sourced from mammalian milk.

In alternative embodiments of the instant invention, the MMO (e.g., bovine milk oligosaccharides (BMO) or human milk oligosaccharides (HMO)) may be supplemented with synthetically-produced oligosaccharides including fucosyllactose (SPF) and/or sialyllactose (SPS), or more complicated structures such as, but not limited to, 2′-fucosyllactose, 3-fucosyllactose, difucosyllactose, lacto-N-fucosylpentaose I, lacto-N-fucosylpentaose II, lacto-N-fucosylpentaose III, lacto-N-fucosylpentaose V, 3′-sialyllactose, 6′-sialyllactose, 3′-sialyl-3-fucosyllactose, sialyllacto-N-tetraose, N-acetylgalactosamine, and 6′-sialyllactosamine, which have been synthetically produced and may be purified to at least 50% purity before addition to the MMOs. The definition of synthetically produced oligosaccharides in this invention includes those oligosaccharides produced in genetically modified organisms as well as through chemi-synthetic processes that are otherwise identical to MMOs as well as galactooligosaccharides (GOS) that are enriched in DP-4 and DP-5 polymers as described in U.S. Pat. No. 8,425,930 (incorporated here by reference in its entirety) as these structures also provide differential growth of B. infantis. In a preferred embodiment, the synthetically-produced derivatives can be used alone or added to the milk-sourced MMO and make up from at least 5% to at least 80% of the dry weight of the composition. In some embodiments of the present invention, the mass ratio of MMO:SPF or MMO:SPS is from 20:1 to 1:5, in a preferred embodiment the mass ratio of MMO:SPF or MMO:SPS is from 10:1 to 1:2, and in a most preferred embodiment the mass ratio of MMO:SPF or MMO:SPS is from 5:1 to 1:1. Ratio targets may also be that of human milk wherein one starts with Sialyllactose-dominant compositions such as bovine milk and add one or more of purified 2′-fucosyllactose, 3-fucosyllactose, difucosyllactose, lacto-N-fucosylpentaose I, lacto-N-fucosylpentaose II, lacto-N-fucosylpentaose III, lacto-N-fucosylpentaose V, lacto-N-tetrose, and lactose-N-neotetrose.

### Affecting Intestinal Mucosa Beyond the Neonatal Stage

The instant invention can be used to treat a mammalian infant or non-infant patient (beyond 6 months of age), where the patient has a gastrointestinal distress caused by elevated levels of pathogenic bacteria (dysbiosis) such as, but not limited to, Listeria, Chlamydia, Escherichia, Helicobacter, Shigella, Salmonella, Yersinia, Clostridium, Campylobacter, and other members of the Proteobacteria which can damage the gut epithelium and mucosa. Such dysbioses include, but are not limited to, Irritable Bowel Syndrome (IBS), Crohn's Disease (CD), and Ulcerative colitis (UC); (collectively IBD) Necrotizing Enterocolitis (NEC), bacterial overgrowth (BO), bacterial induced diarrhea (BID), Celiac Disease (CEL), and antibiotic treatment (AT). In some embodiments of the present invention the dysbiosis may be defined by a less complex and/or less abundant microbiome than normal which may be due to causes including, but not limited to, prolonged antibiotic treatments, narrow dietary diversity, and eating disorders, such as, but not limited to, bulimia nervosa, anorexia nervosa, and binge eating disorder.

In this invention, treatment of the gastrointestinal distress is provided with an oral dose of bacteria such as, but not limited to bifidobacteria, and MMOs including, but not limited to milk oligosaccharides from a mammalian source, MMOs from other biological sources, or chemically or biologically synthesized MMOs that are the functional equivalent of those found in mammalian milk sources, and GOS polymers enriched in DP-4 and DP-5.

In some embodiments, any of the compositions described herein can be provided to a non-nursing mammal. The non-nursing mammal can be a human, as well other domesticated mammalian species such as, but not limited to, an agriculturally-relevant production mammal (e.g., cow, pig, rabbit, goat, buffalo, and sheep), a mammalian companion animal (e.g., cat, dog, rabbit, and horse), laboratory mammals (e.g., mice and rats), and performance mammals (e.g., a thoroughbred race horse, camel, and working dog).

In some embodiments, a composition comprising a Bifidobacterium and a MMO is provided. The Bifidobacterium can be B. longum (e.g., B. longum subsp. infantis, B. longum subsp. longum), B. breve, B. bifidum, B. animalis (e.g., B. animalis subsp lactis, B. animalis subsp animalis), B. pseudocatenulatum, B. adolescentis, B. catenulatum, B. pseudolongum, or any combination thereof. In some embodiments, the composition provides a mucosal healing to a mammal by acting as an anti-inflammatory to sooth intestinal inflammation caused by dysbiosis or other disease and also preventing the growth and thereby removing the unwanted or overgrown bacteria. The composition, when provided to a mammal, may allow for colonization by the bifidobacteria and displacement of other bacteria. When the composition is administered and, optionally, combined with a regulated diet, the microbiome can have reduced numbers of non-bifidobacteria species as compared to a microbiome of one not being administered the composition. In one embodiment, administration of the composition results in a “simple microbiome” due to the increased proportional colonization by the bifidobacteria.

A simple microbiome can be described as the presence of greater than 10⁶ cfu/g stool of a single genus of bacteria (e.g., Bifidobacterium), more particularly, of a single species or strain of bacteria (e.g., B. longum subsp. infantis [B. infantis]). This can be reflected in, for example, up to 80% of the microbiome being dominated by the bacterial genus or, more particularly, by the single subspecies of a bacteria such as B. infantis in a human breast feeding infant. A simple microbiome can also be described as the presence of greater than 10%, 20%, 30%, 40%, 50%, 60%, 70%, 75%, 80%, or 90% of a single genus of bacteria (e.g., Bifidobacterium), more particularly, of a single subspecies as a percent of total bacterial cells (e.g., B. longum subsp. infantis [B. infantis]) in animals, or in other patient groups

In a preferred embodiment the bifidobacteria is selected from B. longum, B. breve, B. bifidum, B. animalis subsp lactis, B. animalis subsp animalis, B. pseudocatenulatum, B. catenulatum, or any combination thereof. In a more preferred embodiment the bifidobacteria is selected from a group of bifidobacteria that internalize mammalian milk oligosaccharides prior to their hydrolysis such as, but not limited to B. longum, B. breve, and B. pseudocatentulatum. In a particularly preferred embodiment of the invention, the bifidobacteria is B. longum subspecies infantis.

Additional embodiments involve the feeding of a mammal of any age in need of mucosal development or healing with a composition comprising bifidobacteria (e.g., activated bifidobacteria), and a MIO composition. Such a composition can be provided at a dose level of from 10 million to 1 trillion cfu/day of bifidobacteria, and from 1 to 60 g/day of MIO composition for a period of from 1-60 days. A mammal (e.g., human) in need of mucosal healing would include, but would not be limited to, individuals with signs or symptoms of NEC, IBS, IBD, Crohn's Disease, leaky gut, auto inflammatory diseases, autism, obesity, asthma, food allergies, eating disorders, or pathogenic bacterial overgrowth, as well as individuals that have had a course of antibiotic therapy and are repopulating their GI tract.

In other embodiments of the instant invention the treatment of the gastrointestinal distress is provided by an oral dose of bifidobacteria described above that can internalize and consume MMO such as HMO or BMO, along with commensal bacteria that can consume free sugar monomers, where such commensal bacteria are preferably Lactobacillus and/or Pediococcus species that selectively consume monomer sugars such as, but no limited to, fucose and/or sialic acid.

In some embodiments of the instant invention, the MMO, bifidobacteria, and optionally lactobacilli are provided to a patient together in a dry powder form or encapsulated in a two-part capsule enrobed with an enteric coating, and provided to a patient in need of such treatment at a dose of from 10 million to 1 trillion cfu of bifidobacteria and 10 million to 100 billion cfu of lactobacilli per day and from 1 to 60 g of MMO per day. In a more preferred embodiment, the MMO and probiotic bacteria are provided to a patient at a dose of from 4 to 50 Billion cfu of bifidobacteria plus 4-50 Billion cfu of lactobacilli per day and from 2 to 30 g of MMO per day. In a particularly preferred embodiment the Bifidobacterium is B. longum subsp. infantis, and the Lactobacillus is L. reuteri.

In various embodiments of the invention, the composition comprising the bifidobacteria and a MMO selected from MMO, SPF and/or SPS and/or GOS, is provided to a mammal (e.g., a human) in order to overcome gut-related disorders in obesity including, but not limited to, gut-related metabolic disorders such as hyperphagia and Type I and Type II diabetes by any of a number of mechanisms including, but not limited to, the restoration of gut barrier function and the reduction of food intake. This embodiment includes all ages of mammals (e.g., humans) including newborn infants, children, adolescents, adults, and geriatric mammals (e.g., humans).

In some embodiments of the instant invention, the combination of (a) bifidobacteria capable of internalizing a MMO prior to hydrolysis and (b) a MMO such as, but not limited to BCO, BMO, HMO, SPF and/or SPS, and/or GOS is provided to a human or mammalian patient exhibiting a dysbiosis-related intestinal pathology. Such a treatment is maintained on a daily basis until the concentration of the bifidobacteria achieves at least a 2-fold increase in the numbers of the bifidobacteria in the gut of the mammal. In a preferred embodiment the levels reach at least a 10-fold increase. In a more preferred embodiment the levels reach at least a 100-fold increase. In a preferred embodiment of the invention the patient will receive essentially no other oligosaccharide or dietary fiber other than the delivered BCO, BMO, HMO, SPF and/or SPS and/or GOS during the treatment period. Although optional, it may be beneficial to “clean out” the intestine prior to treatment, typically by use of laxatives to encourage expulsion of any residual fiber present prior to treatment. In certain embodiments, the bifidobacteria capable of internalizing a mammalian milk oligosaccharide prior to hydrolysis is an activated bifidobacteria. An activated bifidobacteria is a bifidobacteria that, through contact with milk glycans has genes of an HMO gene cluster that are upregulated. In certain other embodiments, the bifidobacteria capable of internalizing a MMO prior to hydrolysis is cultured in a manner that is non-activating.

In certain embodiments of the instant invention, a “daily ration” of the bifidobacteria and MMO is provided to the patient. A “daily ration” is an amount provided to the patient within the same 24-hour period. A patient can be given a dose of the bifidobacteria and a dose of the MMO substantially contemporaneously (e.g., within six hours, within four hours, within two hours, within one hour, within forty-five minutes, within thirty minutes, within twenty minutes, within fifteen minutes, within ten minutes, within five minutes, within three minutes, or within one minute).

In some embodiments of the instant invention, the dosing of the bifidobacteria and MMO is maintained for a period of at least 1 week to allow mucosal healing. In a more preferred embodiment the dosing of the bifidobacteria and MMO is maintained for a period of at least 1 month to allow full mucosal healing. In a particularly preferred embodiment the dosing of the bifidobacteria and MMO may be continued through the period of time during which the symptoms of the intestinal pathology are alleviated. Preferably, dosing is discontinued when GI symptoms have been alleviated, and the patient is able to transition without symptoms to adult dietary sources of fibers using a strategy of weaning away from a single fiber source to multiple fiber sources supported with commensal organisms adapted to the adult dietary fiber. Such a weaning process is described in U.S. Patent Application No. 62/307,425, which is incorporated herein by reference in its entirety. Dosing may be continued while the symptoms are alleviated for a period of time (e.g., one hour, two hours, three hours, four hours, six hours, eight hours, ten hours, one day, two days, three days, a week, two weeks, at least 1 month, at least from 1 month to 6 months, and at least 6 months to one year).

In various embodiments of the invention, the high levels of bifidobacteria are returned to normal (low) levels by eliminating the dietary supply of MMO, BCO, BMO, HMO, SPS, and/or SPF, and GOS, and introducing other conventional food fiber sources as part of the daily diet for a period of at least 1 week. In a preferred embodiment the levels of bifidobacteria are reduced to normal levels by eliminating the dietary supply of MMO, BCO, BMO, HMO, SPS, SPF, and GOS and allowing other conventional food fiber sources as part of the daily for a period of at least 1 month.

Formulating Compositions for this Invention

The MMO preparation may be provided together with preferred bacteria or separately. In a preferred embodiment, the MMO is prepared from a bovine colostrum (BCO), whey permeate or other dairy streams (BMO), and combined with the bifidobacteria at a ratio of from 0.01 to 10 g of MMO per Billion cfu of bifidobacteria. In a more preferred embodiment, the MMO is combined with the bifidobacteria at a ratio of from 0.1 to 1.0 g of MMO per Billion cfu of bifidobacteria.

In a more preferred embodiment the oligosaccharide is provided in a concentrated form, wherein the concentration of the MMO comprises at least 10% of the mass of the preparation (on a dry weight basis) delivered to the human or other mammal in need of the treatment. The preparation may be provided in a dry powder formulation, a solution, a suspension, or in a tablet or capsule format with or without an enteric coating to allow passage through the stomach and release in the intestine. Such enteric coatings include, but are not limited to, dairy proteins, whey proteins fatty acids, waxes, shellac, plastics, plant fibers, methyl acrylate-methacrylic acid copolymers, cellulose acetate succinate, hydroxy propyl methyl cellulose phthalate, hydroxy propyl methyl cellulose acetate succinate, polyvinyl acetate phthalate (PVAP), methyl methacrylate-methacrylic acid copolymers, cellulose acetate trimellitate, sodium alginate, and Zein.

Administering the Composition of this Invention

In some embodiments of the instant invention, the MMO and bifidobacteria are provided to a patient together in a dry powder form and/or encapsulated in a two-part capsule enrobed with an enteric coating, and provided to a patient in need of such treatment at a dose of from 10 million to 100 billion cfu of bacteria per day and from 1 to 60 g of mammalian milk oligosaccharides per day. In a more preferred embodiment, the mammalian milk oligosaccharide and bacteria are provided to a patient at a dose of from 4 billion to 50 billion cfu of bacteria per day and from 2 to 40 g of MMO per day.

In some embodiments the oligosaccharide component can be dissolved in a liquid such as, but not limited to, water, physiological saline, mammalian milk, or formulation designed to provide all or part of a daily nutritional requirement such as, but not limited to an infant formula or an enteral formula and provided in a liquid form to the patient while the bifidobacteria are provided separately as a powder or suspension in a carrier liquid which may optionally include a solution comprising the MMO.

In some embodiments of the invention, the patient is maintained on a strictly controlled diet throughout the course of the treatment with the bacteria and MMO. Such a diet would contain none or a minimal amount of any other dietary fiber but may contain simple carbohydrates such as monosaccharides and disaccharides in amounts required to maintain the patients pre-intervention weight. In a preferred embodiment of the instant invention, the daily amount of the other dietary fiber is from less than 30 g/day, preferably less than 10 g/day, more preferably less that 5 g/day, and most preferably less than 1 g/day. In a preferred embodiment of the instant invention, the dietary simple carbohydrates are less than 50 g/d. In a preferred embodiment of the instant invention, the daily amount of the simple carbohydrates is less than 40 g/d, preferably less than 20 g/day, more preferably less than 10 g/day, and most preferably less than 5 g/day.

In other embodiments the oligosaccharide component and the bifidobacteria are provided together in a spoonable composition such as, but not limited to, yogurt, kefir, pudding, cream, chocolate, or any edible oil.

In some embodiments of the invention, any of the compositions described herein may be administered to a patient. Patients include mammals suffering from gut-related disorders including, but not limited to, obesity, or gut-related metabolic disorders such as hyperphagia and Type I and Type II diabetes, by any of a number of mechanisms including, but not limited to, the restoration of gut barrier function and the reduction of food intake. Mammals may include humans, as well as other domesticated mammalian species including, but not limited to, agriculturally-relevant production mammals (e.g., cows, pigs, rabbits, goats, and sheep), mammalian companion animals (e.g., cats, dogs, and horses), and performance mammals (e.g., thoroughbred race horses, racing camels, and working dogs). Patients may include all ages of mammals including infant mammals, young mammals, adolescent mammals, adult mammals, and geriatric mammals.

Those who would particularly benefit from the process of this invention include patients with a bacterial bloom that rapidly expands the presence of a particular organism, or patients with reduced diversity where key commensal species are missing. Both of these cases may present as a microbiome of less diversity than expected in a healthy individual, and these patients are characterized as having a dysbiotic microbiome. Shifts in the microbiome can be determined using Next Generation Sequencing (see, e.g., Ji et al., “From next-generation sequencing to systematic modeling of the gut microbiome”, Front Genet. (Jun. 23, 2015), published online at doi.org/10.3389/fgene.2015.00219) or full Metagenomics approaches (see, e.g., Wang et al., “Application of metagenomics in the human gut microbiome”, World J. Gastroenterol. (2015), Vol. 21, No. 3, pp. 803-814) to monitor the change in specific organisms, or overall shifts in families known to contain members of opportunistic or pathogenic organisms. qPCR can also be used to monitor changes in specific species or subspecies. Typically measurements can be normalized using the amount of DNA per gram of stool. A simple microbiome may be healthy in the case of an infant whose diet is almost entirely composed of a single nutrient source (e.g., mother's milk). However, for an individual consuming a more varied diet, a shift of the microbiome to simpler structure is typically an indication of dysbiosis.

In some embodiments, a patient is administered a composition comprising bifidobacteria and an oligosaccharide component for a period of time, following which the patient is administered a composition comprising an oligosaccharide component that does not comprise bifidobacteria to keep the bifidobacteria colonized.

In other embodiments, a patient is administered a composition comprising bifidobacteria for a period of time, following which the patient is administered a composition comprising bifidobacteria and an oligosaccharide component.

In some embodiments, a patient is administered a composition comprising bifidobacteria and an oligosaccharide component for a period of time, following which the patient is administered a composition comprising bifidobacteria that does not include an oligosaccharide component.

In some embodiments, a patient is administered an oligosaccharide component for a period of time, following which the patient is administered a composition comprising bifidobacteria and an oligosaccharide component. The initial oligosaccharide component can be provided in an amount that provides at least 1 g per day of MMO to the patient. For example, the initial oligosaccharide component can be provided in an amount that provides at least 1 g per day, at least 3 g per day, at least 5 g per day, at least 8 g per day, at least 10 g per day, at least 15 g per day, at least 20 g per day, at least 25 g per day, at least 30 g per day, at least 35 g per day, at least 40 g per day, at least 50 g per day, or at least 60 g per day of mammalian milk oligosaccharides to the patient.

In various embodiments, a patient is administered a composition comprising bifidobacteria and/or an oligosaccharide component for a period of time, following which the patient is administered a composition comprising bifidobacteria and/or an oligosaccharide component for a period of time in which the amount administered is tapered (e.g., administered at a generally decreasing rate) for a second period of time.

Certain embodiments of the invention involve a combination of a composition comprising MMO and/or SPF and/or SPS and/or GOS and a bifidobacteria wherein the Bifidobacterium is selected from B. longum, B. breve, B. bifidus, B. animalis subsp lactis, B. animalis subsp animalis, B. pseudocatenulatum and B. catenulatum or any combination thereof. In a more preferred embodiment the Bifidobacterium is B. longum subp infantis.

In other embodiments of the invention, the Bifidobacterium species is used in combination with a Lactobacillus species including, but not limited to, L. plantarum, L. antri, L. brevis, L. casei, L. coleohominis, L. fermentum, L. gasseri, L. johnsonii, L. pentosus, L. sakei, L. salivarius, L. rhamnosus (e.g., LGG), L. acidophilus, L. curvatus, and L. reuteri. In a preferred embodiment the composition comprises MMO and/or SPF and/or SPS, and/or GOS, or derivatives thereof, L. rhamnosis and B. longum subsp. infantis.

### EXAMPLES

### Example 1. Preparation of Human Milk Oligosaccharide (HMO) Compositions that can be Used Exclusively by Certain Bifidobacteria

A concentrated mixture of HMO is obtained by a process similar to that described by Fournell et al (US Patent Application 2015/0140175). Human milk is pasteurized and then centrifugally defatted, separating it into cream (predominantly fat) and skim milk (defatted product). The defatted skim milk is then ultrafiltered using membranes with a 5-10 kDa cut off to concentrate a protein fraction (predominantly whey proteins and caseins). The permeate from the ultrafiltration, comprising lactose and the complex HMOs, is dried directly by spray drying, or the lactose is partially eliminated by an additional ultrafiltration using a 1 kDa cut off filter before drying. The composition of this dried fraction is typically about 50% lactose and about 30% mammalian milk oligosaccharides (HMO) with the remainder of the mass primarily peptides and ash. The HMO fraction is predominantly fucosylated. However, these compositions can vary from 20-70% lactose and 10-50% mammalian milk oligosaccharides (HMO) depending on the ultrafiltration processes.

### Example 2. Preparation of Bovine Milk Oligosaccharide (BMO) Compositions that can be Used Exclusively by Certain Bifidobacteria

A concentrated mixture of bovine milk oligosaccharide (BMO) was obtained from whole milk which was pasteurized by heating to 145 degrees F. for 30 minutes, cooled and centrifugally defatted, separating it into cream (predominantly fat) and skim milk (defatted product). The defatted skim milk was then ultra-filtered using membranes with a 5-10 kDa cut off to concentrate a protein fraction (predominantly whey, proteins and caseins). The lactose in the permeate was partially eliminated by an additional nanofiltration using a 1 kDa cut off. The composition was then spray dried. This composition of dried BMOs comprised about 15% lactose and about 10% BMO with the remainder of the mass primarily peptides, ash and other components. Twenty grams of this composition was combined with 5 g of GOS (Vivinal GOS) as the daily ration for treatment.

### Example 3. Preparation of Bovine Colostrum Oligosaccharide (BCO) Compositions that can be Used Exclusively by Certain Bifidobacteria

A concentrated mixture of BCO is obtained by a process such as that described by Christiansen et al (2010) International Dairy Journal, 20:630-636. Bovine colostrum (preferably from the first milking) is pasteurized by heating to 145 degrees F. for 30 minutes, cooled and centrifugally defatted, separating it into cream (predominantly fat) and skim milk (defatted product). The defatted skim milk is then ultra-filtered using membranes with a 5-10 kDa cut off to concentrate a protein fraction (predominantly whey, proteins and caseins). The permeate, comprising the lactose and mammalian milk oligosaccharides, is dried directly by spray drying. Alternatively, the lactose is partially eliminated by an additional nanofiltration using a 1 kDa cut off. The composition of this dried oligosaccharide fraction is about 40% lactose and about 40% bovine colostrum oligosaccharides (BCO) with the remainder of the mass primarily peptides and ash. The BCO fraction is predominantly sialylated.

### Example 4. Preparation of an Activated Bifidobacteria Composition that can Exclusively Use Certain Mammalian Milk Oligosaccharides

Bifidobacterium longum subsp infantis was isolated and purified from the feces of a vaginally delivered, breast fed human infant, and its identification was confirmed by DNA analysis that reflected the presence of a gene set that is specifically associated with this organism (Sela et al., 2008, PNAS, 105:18964-18969). A seed culture of this organism was added to a standard growth medium comprising glucose and the BCO of Example 3 as carbon sources in a 500 L agitated fermenter. Following 3 days of growth under anaerobic conditions, a sample of the culture was tested for the presence of activated Bifidobacterium longum subsp. infantis. Activated B. infantis was identified by the presence of gene transcripts for sialidase. The fermenter was harvested by centrifugation, the concentrated cell mass was mixed with a cryopreservative (trehalose plus milk proteins) and freeze dried. The final dry product was 5.5 kg of bacterial mass with a live cell count of 130×10⁹ cfu/g.

### Example 5. Preparation and Use of Therapeutic Compositions for the Treatment of Digestive Pathologies

The activated B. infantis product of Example 4 was blended with pharmaceutical grade lactose to provide a minimum dose of 30 Billion cfu of B. longum subsp. infantis per gram. 0.625 g of this diluted activated B. infantis product was then packaged in oxygen- and moisture-resistant sachets, to provide doses of 15 Billion cfu of B. longum subsp. infantis per sachet. One sachet of 18 billion cfu of B. longum subsp. infantis was consumed with a morning breakfast and one with an evening meal.

Twenty grams of the BMO preparation of Example 2 was combined with five g of GOS, packaged in separate bags and administered in a daily ration of 20 g BMO+5 g GOS. This preparation provided the carbon source (BMO and GOS) to support the specific growth of the supplemented B. longum subsp. infantis in the colon of the patient, thereby providing a gut environment favoring mucosal healing.

The BMO preparation was consumed 5 times per day (5×5 g BMO/GOS mixture of Example 2), approximately every 3-4 hr by blending the 5 g of powder with a meal replacer (Boost, Nestle Nutrition) containing 240 Cal/drink with 15 g/protein and 6 g of fat and 0 g of dietary fiber. The subject was allowed to consume 2-3 eggs each morning, and one serving of fish or meat with lunch and dinner. Any dietary fiber consumption outside the therapeutic formulation of BMO was kept at less than 1 g per day.

As a step to accelerate the switch from a microbiome consuming adult dietary fiber to a microbiome consuming milk-based fiber, the subject completed a colonoscopy preparation involving a clear liquid diet and laxatives to clear out the bowels of fiber in preparation for the diet change. Once this was completed, the subject followed the specific diet regimen that limited the non-milk dietary fiber to less than 1 gram per day and ensured the subject was still eating a diet with sufficient protein, fat and carbohydrate to maintain a constant weight.

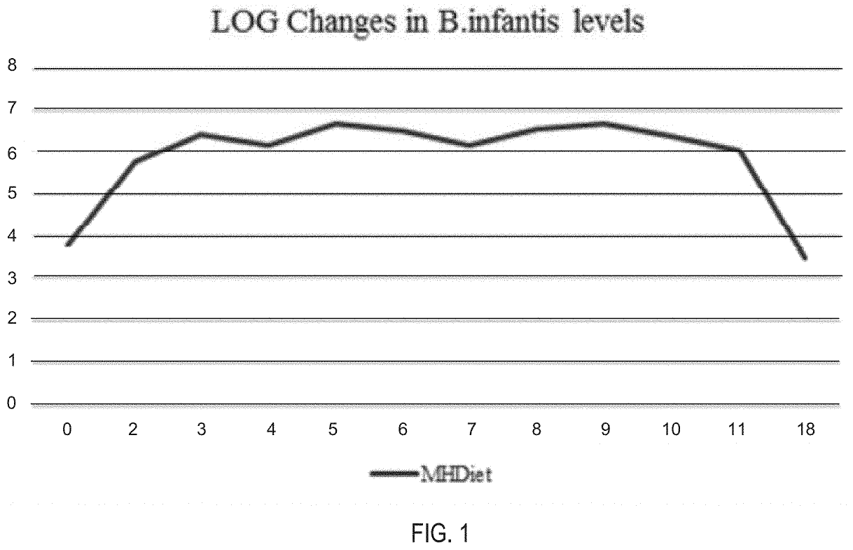

Fecal samples were taken the day before the colonoscopy prep (pretreatment) and on a daily basis for the 7 days on the dietary regimen of consumption of the B. infantis and BMO. The subject also filled out questionnaire forms regarding a self-assessment of the subject's gastrointestinal responses or indicators of the palliative effect of the composition on symptoms of gastrointestinal distress. Following the seven days of the dietary regimen, the subject was allowed to return to his pretreatment standard diet and post treatment fecal samples were taken during a 1 week post-treatment phase. DNA was extracted and subjected to qPCR analysis and NextGen sequencing for microbiome analysis. B. infantis was specifically measured using qPCR (FIG. 1). At baseline, B. infantis was below the limit of detection in an adult gut. Detectable levels were observed with supplementation and diet changes. FIG. 1 shows that there was at least a 1,000-fold difference in levels of colonic B. infantis between baseline and treatment. The NGS data provided a means of visualizing the relative changes in different clades and families of bacteria. Samples were also prepared for other measurements including BMO content by Mass Spectrometry in the stool to monitor in vivo consumption, short chain fatty acid and lactate, pH determinations, measurements of cytokines and a full metabolomics determination.

TABLE 1
Study Schedule
BMO/GOS Treated Participant

| | D 0 | D 1 | D 2 | D 3 | D 4 | D 5 | D 6 | D 7 | D 8 | D 9 | D 10 | D 11 | D 18 |
|---|---|---|---|---|---|---|---|---|---|---|---|---|---|
| Colonoscopy Prep | | X | | | | | | | | | | | |
| Regular diet | X | | | | | | | | | X | X | X | X |
| No fiber diet | | X | X | X | X | X | X | X | X | | | | |
| BMO & GOS | | X | X | X | X | X | X | X | X | | | | |
| B. infantis | | | X | X | X | X | X | X | X | | | | |
| (1AM & 1PM) | | | | | | | | | | | | | |
| Swab (x2) | X | | X | X | X | X | X | X | X | X | X | X | X |
| Stool | X | | X | X | X | X | X | X | X | X | X | X | |

### Example 6. Use of a Composition of B. longum Subsp. Infantis with Lactobacillus plantarum to Reduce Clostridium Species in Newborn Foals

Newborn foals born to mares at a large horse breeding barn were monitored during an outbreak of severe hemorrhagic diarrhea among the foals. The foals were found to be culture- and toxin-positive for Clostridium difficile. Seventeen foals were born during the initial phase of the outbreak, of which fifteen animals became ill and required intervention according to the standard of care as described in the Merck Veterinary Manual. Standard of care involved metronidazole treatment given at a dose of 15-20 mg/kg, PO, tid-qid. and may also involve administration of large volumes of interveneous polyionic fluids, with supplemental electrolytes (potassium, magnesium, and calcium), plasma or synthetic colloids for low oncotic pressure, anti-inflammatories such as flunixin meglumine, and broad-spectrum antibiotics if the horse is leukopenic and at risk of bacterial translocation across the compromised GI tract. Polymyxin B may aid in binding systemic endotoxin.

Of these seventeen foals, fifteen developed loose stool or diarrhea lasting 3-4 days, and 2 died as a result of the infection. After observing the outbreak, the next foals were provided a formulation of of 3×10¹² CFU Bifidobacterium longum subsp. infantis EVBL001 and 5×10⁹ CFU of Lactobacillus plantarum EVLP001 every 12 hours. The two foals that were provided with the formulation at 12 hours of age developed a mild diarrhea, but recovered within 8 hours compared to 3-4 days with standard of care. None of the foals provided with this dose starting at birth developed diarrhea (n=6).

Recovery time for the two treated animals that eventually developed the infection was approximately eight hours, which was significantly shorter than the normal recovery time of at least 3-4 days for animals given the standard care regimen. No adverse events were recorded among the treated animals and the dosages were well tolerated. A Fisher's exact test of the two populations (Standard of Care and Probiotic treated) yields a significant difference in incidences of C. difficile infection (p=0.0016) (Table 2).

TABLE 2
Fisher's Exact Test
The two-tailed P value equals 0.0036

| | Healthy | Diarrhea | Total |
|---|---|---|---|
| Control | 2 | 15 | 17 |
| Treated | 6 | 2 | 8 |
| Total | 9 | 17 | 26 |

A 2 × 2 Contingency table analyzed by Fisher's Exact test indicates a significant reduction in sick animals among those treated with the probiotic mixture (Treated), relative to the standard of care (Control).

Two treatment options were attempted. In the first, animals were dosed at 12 hours of life, but this fails to significantly reduce incidence of diarrhea (given the small n), though the severity (duration) was dramatically shortened to 12 hours or less (p=0.0074; Fisher exact test, comparing populations of diarrheal foals segregated by duration of diarrhea). The second option, dosing at birth, was significant at reducing incidences of diarrhea (p=0.0025). All animals were dosed at birth with 6.6 mg/kg of ceftiofur (Excede), and this did not affect health outcome, related to diarrhea. Additionally, the treated population did not develop foal heat diarrhea, which typically affects >50% of animals, and requires treatment in approximately 10% of cases (Weese and Rousseau 2005). If a >50% risk is extrapolated to a hypothetical population of 8 animals to match the 8 observed; this yields a significant reduction in foal heat diarrhea (p=0.0256).

The results described above demonstrate that administration of a composition that includes Bifidobacteria (e.g, B. longum subspecies infantis) with a Lactobacillus (e.g., L. plantarum) that was chosen to consume the free sugar monomers that a known pathogen (e.g., a Clostridium species) preferred to consume was effective at reducing the dysbiotic episodes for the newborn foals. This example is not limited to newborn foals, but demonstrates that administration of the compositions described herein can be effective to reduce or eliminate dysbiotic episodes in mammals. While this example provides experimental support for the concept underlying this invention, it should be noted that these foals were nursing animals where MMO was supplied by mother's milk. Therefore, a supplemental MMO was not provided. Similar results would be expected, were the MMO to be provided as a supplement, rather than mother's milk.

### Example 7. Providing B. infantis to Nursing Piglets

In untreated young nursing pigs, populations of Enterobacteriaceae in the gut were found to correlate with the abundance of Bacteroides (r2=0.661, p<0.001). It was also found that these populations of Enterobacteriaceae cannot, by themselves, consume sialylated pig milk oligosaccharides, but Bacteroides possess enzymes capable of releasing sialic acid from pig milk oligosaccharides, which is associated with increased abundances of sialic acid in feces. Enterobacteriaceae can consume the sialic acid released by Bacteroides. The treatment of pigs with Bifidobacterium and/or Lactobacillus reduced the amount of sialic acid available and resulted in a reduction in scours (See WO 2016/094836 & WO 2016/149149, the disclosures of which are incorporated herein in their entirety).

### Example 8. Increase in Weight Gain in Nursing Piglets Fed a Prebiotic Composition

Pig litters are typically given antibiotics prophylactically at birth to prevent early infections during nursing including scours. Scours can be infectious from viral or bacterial causes (most are viral) or can be associated with early post-weaning. Scours is detrimental to the overall performance and health of the pig.

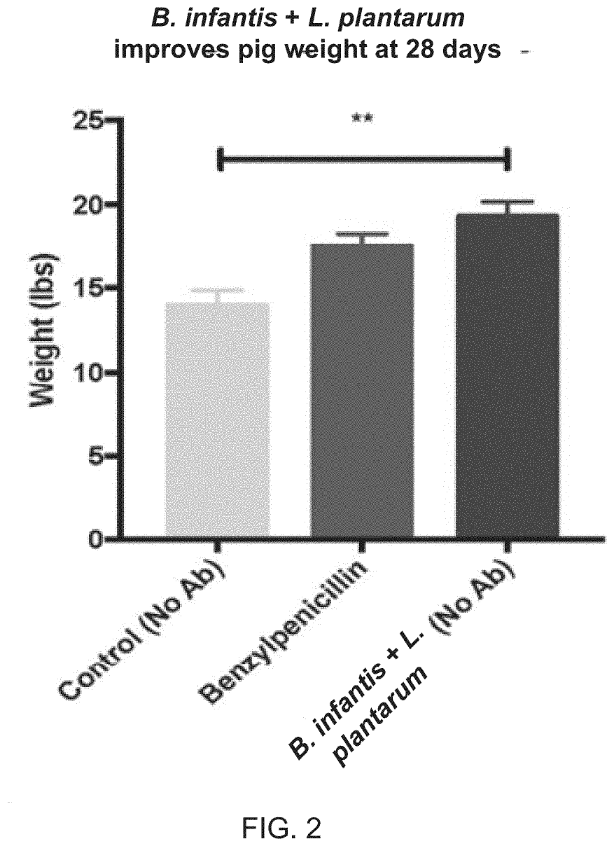

Several litters were randomized into one of three groups. One group received a standard of care dose of benzylpenicillin at birth, another group received no benzylpenicillin, and another group received no benzylpenicillin but was given 18 billion CFU of activated B. infantis EVC001 (Example 4) and 1 billion CFU of L. plantarum EVLP001 daily by oral gavage for seven days, from day 14-21 of life. L. plantarum EVLP001 was isolated from the feces of a nursing piglet and cultured in a food-grade sterile milk medium, without stirring, at 37 C and enumerated on MRS medium to confirm dosing. All animals were weighed at 28 days of life and compared across treatment groups. As shown in FIG. 2, the piglets that received B. infantis at 14-21 days had the highest weight gain.

1. A medicament for treating a patient with intestinal distress comprising a mammalian milk oligosaccharide and a bifidobacteria that internalizes said mammalian milk oligosaccharide prior to its hydrolysis.

2. The medicament of claim 1, wherein the intestinal distress represents a microbial dysbiosis.

3. The medicament of claim 1, wherein the intestinal distress is the result of Irritable Bowel Disease, Crohn's Disease, Ulcerative colitis, Necrotizing Enterocolitis, bacterial or viral overgrowth, bacterial induced diarrhea, antibiotic treatment, eating disorders, autism, obesity, or low diversity in dietary intake.

4. The medicament of any one of claims 1-3, wherein the bifidobacteria is selected from B. longum, B. animalis, B. catenulatum, B. pseudolongum, B. pseudocatanulatum, and B. breve.

5. The medicament of claim 4, wherein the B. longum is B. longum subs. infantis.

6. The medicament of any one of claims 1-5, wherein the mammalian milk oligosaccharide is from mammalian milk.

7. The medicament of claim 6, wherein the mammalian milk is from human, bovine, or caprine sources.

8. The medicament of claim 7, wherein the bovine source is from bovine colostrum.

9. The medicament of claim 6, wherein the mammalian milk oligosaccharide is from whey permeate.

10. The medicament of any one of claims 1-9, wherein the mammalian milk oligosaccharide comprises fucosyllactose, sialyllactose or derivatives thereof.

11. The medicament of any one of claims 1-10, wherein the mammalian milk oligosaccharide comprises one or more carbohydrates selected from synthetically produced and purified 2′-fucosyllactose, 3-fucosyllactose, difucosyllactose, lacto-N-fucosylpentaose I, lacto-N-fucosylpentaose II, lacto-N-fucosylpentaose III, lacto-N-fucosylpentaose V, 3′-sialyllactose, 6′-sialyllactose, 3′-sialyl-3-fucosyllactose, sialyllacto-N-tetraose, N-acetylgalactosamine, and 6′-sialyllactosamine

12. The medicament of any one of claims 1-11, wherein the medicament further comprises Lactobacillus and/or Pediococcus.

13. The medicament of claim 12, where in the Lactobacillus is selected from L. plantarum, L. antri, L. brevis, L. coleohominis, L. fermentum, L. gasseri, L. johnsonii, L. pentosus, L. sakei, L. sahvarius, L. casei, L. rhamnosus (e.g., LGG), L. acidophilus, L. curvatus, L. reuteri, L. mucosae, and L. crispatus

14. The medicament of claim 13, wherein the Lactobacillus is L. reuteri.

15. The medicament of any one of claims 12-14, wherein the Lactobacillus is provided in a daily dose of from 10 million to 1 trillion cfu.

16. The medicament of any one of claims 12-15, wherein the Lactobacillus is provided in a daily dose of from 10 billion to 50 billion cfu.

17. The medicament of any one of claims 1-16, wherein the mammalian milk oligosaccharide is in the form of a powder.

18. The medicament of any one of claims 1-17, wherein the medicament is in the form of a powder.

19. The medicament of any of claims 1-18, wherein the oligosaccharide is formulated to provide dietary fiber in an amount suitable for a non-infant patient.

20. The medicament of claim 19, wherein the non-infant patient is at least 6 months of age.

21. The medicament of any one of claims 1-20, wherein the medicament is formulated to provide a daily ration sufficient to support the intestinal microbiome of a patient with a body weight greater than 10 kg.

22. A method of treating gastrointestinal dysbiosis by providing a patient with a composition comprising a mammalian milk oligosaccharide from a mammalian milk source and bifidobacteria that internalizes said mammalian milk oligosaccharide prior to its hydrolysis for at least 5 days.

23. The method of claim 22, wherein the gastrointestinal dysbiosis is associated with Irritable Bowel Disease, Crohn's Disease, Ulcerative colitis, Necrotizing Enterocolitis, bacterial or viral overgrowth, bacterial induced diarrhea, antibiotic treatment, eating disorders, autism, obesity, or low diversity in dietary intake.

24. The method of any one of claim 22 or 23, wherein the bifidobacteria is selected from B. longum, B. pseudocatanulatum, and B. breve.

25. The method of any one of claim 24, wherein the B. longum is B. longum subs. infantis.

26. The method of any one of claims 22-25, wherein the mammalian milk oligosaccharide is from a mammalian milk sourced.

27. The method of any one of claims 22-26, wherein the mammalian milk is from human, bovine, equine, or caprine sources.

28. The method of claim 27, wherein the bovine source is from bovine colostrum.

29. The method of any one of claims 22-28, wherein the mammalian milk oligosaccharide is from whey permeate.

30. The method of any one of claims 22-29, wherein the mammalian milk oligosaccharide is selected from fucosyllactose, sialyllactose, combinations thereof, and derivatives thereof.

31. The method of any one of claims 22-30, wherein the mammalian milk oligosaccharide is selected from synthetically produced and purified 2′-fucosyllactose, 3-fucosyllactose, difucosyllactose, lacto-N-fucosylpentaose I, lacto-N-fucosylpentaose II, lacto-N-fucosylpentaose III, lacto-N-fucosylpentaose V, 3′-sialyllactose, 6′-sialyllactose, 3′-sialyl-3-fucosyllactose, sialyllacto-N-tetraose, N-acetylgalactosamine, 6′-sialyllactosamine, and combinations thereof.

32. The method of claim 22-27, wherein the mammalian milk oligosaccharide is from human sources.

33. The method of any one of claims 22-25, wherein the mammalian milk oligosaccharide comprises GOS enriched in DP4 and DP5.

34. The method of any one of claim 22-25 or 33, wherein the mammalian milk oligosaccharide is from a recombinant microorganism.

35. The method of any one of claim 22-25 or 33, wherein the mammalian milk oligosaccharide is produced by chemical synthesis.

36. The method of any one of claims 22-35, wherein the bifidobacteria is provided in a daily dose of from 1 million to 100 billion cfu.

37. The method of any one of claims 22-36, wherein the bifidobacteria is provided in a daily dose of from 10 billion to 50 billion cfu.

38. The method of any one of claims 22-37, wherein the mammalian milk oligosaccharide is provided in a daily dose of from 1 to 20 g.

39. The method of any one of claims 22-38, wherein the mammalian milk oligosaccharide is provided in a daily dose of from 1 to 10 g.

40. The method of any one of claims 22-39, wherein the mammalian milk oligosaccharide and the bifidobacteria are present in a dry form and enrobed in a material that would provide enteric protection.

41. The method of any one of claims 22-39, wherein the mammalian milk oligosaccharide and the bifidobacteria are encapsulated and the capsule has an enteric coating.

42. The method of any one of claims 22-39, wherein the mammalian milk oligosaccharide is provided as a solution and the bifidobacteria is provided as an enteric-coated tablet or capsule.

43. The method of any one of claims 22-42, wherein the composition further comprises Lactobacillus and/or Pediococcus.

44. The method of claim 43, wherein the Lactobacillus is selected from L. plantarum, L. casei, L. rhamnosus (e.g., LGG), L. acidophilus, L. curvatus, L. reuteri, L. mucosae, and L. crispatus

45. The method of claim 44, wherein the Lactobacillus is L. reuteri.

46. The method of any one of claims 43-45, wherein the Lactobacillus is provided in a daily dose of from 10 million to 1 trillion cfu.

47. The method of any one of claims 43-46, wherein the Lactobacillus is provided in a daily dose of from 10 to 50 Billion cfu.

48. The method of any one of claims 22-47, wherein the mammalian milk oligosaccharide does not include casein.

49. The method of any one of claims 22-48, wherein the patient is not an infant.

50. The method of any one of claims 22-49, wherein the patient is on a strict diet restricting all other dietary fiber when said composition is provided to said patient

51. The method of any one of claims 22-50, wherein the mammalian milk oligosaccharide is in the form of a powder.

52. The method of any one of claims 22-51, wherein the composition is in the form of a powder.

53. The method of any of claims 22-52, wherein the oligosaccharide is formulated to provide dietary fiber in an amount suitable for a non-infant patient.

54. The method of claim 53, wherein the non-infant patient is at least 6 months of age.

55. The method of any one of claims 22-54, wherein the composition is formulated to provide a daily ration sufficient to support the intestinal microbiome of a patient with a body weight greater than 10 kg.

56. A composition comprising human milk oligosaccharides (HMO) and fucosylated glycans (SPF) and/or sialylated glycans (SPS), wherein the SPF and/or SPS are not of milk origin.

57. The composition of claim 56, wherein the ratio of HMO:SPF is from 20:1 to 1:5.

58. The composition of any one of claim 56 or 57, wherein the composition comprises human milk oligosaccharides and synthetically-produced and purified sialyl glycans (SPS), and wherein the ratio of HMO:SPS is from 5:1 to 1:1.

59. The composition of any one of claims 56-58, wherein the SPF or SPS comprises one or more of 2′-fucosyllactose, 3′-fucosyllactose, difucosyllactose, lacto-N-fucosylpentose I, lacto-N-fucosylpentose II, lacto-N-fucosylpentose III, lacto-N-fucosylpentose V, 3′-sialyllactose, 6′-sialyllactose, 3′-sialyl-3-fucosyllactose, sialyllacto-N-tetraose, and 6′-sialyllactosamine

60. The composition of any of claims 56-59, wherein the composition is formulated to provide dietary fiber in an amount suitable for a non-infant patient.

61. The composition of claim 60, wherein the non-infant patient is at least 6 months of age.

62. The composition of any one of claims 56-61, wherein the composition is formulated to provide a daily ration sufficient to support the intestinal microbiome of a patient with a body weight greater than 10 kg.

63. A composition comprising bovine milk oligosaccharides (BMO) and fucosylated glycans (SPF), wherein the SPF is not of milk origin.

64. The composition of claim 63, wherein the ratio of BMO:SPF is from 20:1 to 1:5.

65. The composition of any one of claim 63 or 64, wherein the composition comprises bovine milk oligosaccharides and synthetically-produced and purified sialyl glycans (SPS), and wherein the ratio of BMO:SPS is from 5:1 to 1:1.

66. The composition of any one of claims 63-65, wherein the SPF or SPS comprises of one or more of 2′-fucosyllactose, 3-fucosyllactose, difucosyllactose, lacto-N-fucosylpentaose I, lacto-N-fucosylpentaose II, lacto-N-fucosylpentaose III, lacto-N-fucosylpentaose V, 3′-sialyllactose, 6′-sialyllactose, 3′-sialyl-3-fucosyllactose, sialyllacto-N-tetraose, N-acetylgalactosamine, and 6′-sialyllactosamine.

67. The composition of any of claims 63-66, wherein the composition is formulated to provide dietary fiber in an amount suitable for a non-infant patient.

68. The composition of claim 67, wherein the non-infant patient is at least 6 months of age.

69. The composition of any one of claims 63-68, wherein the composition is formulated to provide a daily ration sufficient to support the intestinal microbiome of a patient with a body weight greater than 10 kg.

70. A method of treating gastrointestinal dysbiosis comprising administering a mammalian milk oligosaccharide to a patient in need thereof, wherein the patient in need thereof is a patient with a dysbiotic microbiome.

71. The method of claim 70, wherein the dysbiotic microbiome is a bacterial overgrowth of opportunistic pathogens and/or overt pathogens.

72. The method of claim 70 or 71, wherein the dysbiotic microbiome has a reduced microbial diversity.

73. The method of any one of claims 70-72, wherein the mammalian milk is from human, bovine, or caprine sources.

74. The method of claim 73, wherein the bovine source is from bovine colostrum.

75. The method of any one of claims 70-74, wherein the mammalian milk oligosaccharide is from whey permeate.

76. The method of any one of claims 70-75, wherein the mammalian milk oligosaccharide is selected from fucosyllactose, sialyllactose, combinations thereof, and derivatives thereof.

77. The method of any one of claims 70-76, wherein the mammalian milk oligosaccharide is selected from synthetically produced and purified 2′-fucosyllactose, 3-fucosyllactose, difucosyllactose, lacto-N-fucosylpentaose I, lacto-N-fucosylpentaose II, lacto-N-fucosylpentaose III, lacto-N-fucosylpentaose V, 3′-sialyllactose, 6′-sialyllactose, 3′-sialyl-3-fucosyllactose, sialyllacto-N-tetraose, N-acetylgalactosamine, 6′-sialyllactosamine, and combinations thereof.

78. The method of claim 73, wherein the mammalian milk is from human sources.

79. The method of any one of claims 70-78, wherein the patient in need thereof is not an infant.

80. The method of any one of claims 70-79, wherein the mammalian milk oligosaccharide is in the form of a powder.

81. The method of any of claims 70-80, wherein the mammalian milk oligosaccharide is formulated to provide dietary fiber in an amount suitable for a non-infant patient.

82. The method of claim 81, wherein the non-infant patient is at least 6 months of age.

83. The method of any one of claims 70-82, wherein the mammalian milk oligosaccharide is formulated to provide a daily ration sufficient to support the intestinal microbiome of a patient with a body weight greater than 10 kg.
